(19) United States
(12) Patent Application Publication
(10) Pub. No.: US 2019/0174748 A1
(43) Pub. Date: Jun. 13, 2019

(54) COMPOSITION FOR DELIVERY OF ACTIVE AGENTS TO AN ANIMAL

(71) Applicant: Advanced Bionutrition Corp., Columbia, MD (US)

(72) Inventors: Mordechai Harel, Pikesville, MD (US); Swetha Kambalapally, Ellicott City, MD (US)

(73) Assignee: Advanced Bionutrition Corp., Columbia, MD (US)

(21) Appl. No.: 16/278,783

(22) Filed: Feb. 19, 2019

Related U.S. Application Data
(62) Division of application No. 15/735,765, filed on Dec. 12, 2017.
(60) Provisional application No. 62/185,302, filed on Jun. 26, 2015.

(51) Int. Cl. A01N 25/26; A61K 31/573; A61K 9/28; A61K 39/02; A61K 9/50; A61K 9/107; A61K 9/00; A61K 39/112; A61K 39/00; A61K 39/12; A61K 39/116; A01N 25/00

(52) U.S. Cl. CPC A01N 25/26; A61K 2039/55566; A61K 9/282; A61K 9/2813; A61K 39/107; A61K 9/5047; A61K 9/5031; A61K 9/5015; A61K 9/1075; A61K 9/006; A61K 39/0275; A61K 9/2846; A61K 39/0008; A61K 9/288; A61K 9/2866; A61K 39/12; A61K 39/116; A01N 25/004; A61K 9/286; A61K 9/107; A61K 2039/552; A61K 2039/542; A61K 31/573

(57) ABSTRACT

The present invention provides compositions for delivering an active agent to an animal, comprising an active agent, a first coating, a second coating and a third coating. The active agent is coated with the first coating, the first coating is coated with the second coating, and the second coating is coated with the third coating. The active agent is in contact with the first coating, not the second or third coating. The first coating separates the active agent from the second coating while the second coating separates the first and third coatings. The first, second and third coatings are different from each other. Also provided are methods for making and using the compositions.

### CROSS-REFERENCE TO RELATED APPLICATION

This application is a Divisional Application of Ser. No. 15/735,765, filed Dec. 12, 2017, which is a U.S. National Phase Application of International Application No. PCT/US2016/038978, filed Jun. 23, 2016, which claims the benefit of U.S. Provisional Application No. 62/185,302, filed Jun. 26, 2015, the contents of each of which are incorporated herein by reference in their entireties for all purposes.

### BACKGROUND OF THE INVENTION

Administration, especially oral administration, of drugs, vaccines or pesticides offers several advantages. Dosages could be administered to a large number of animals via the food or water with minimal restraint and labor. Restraint also stresses animals rendering the drug or vaccination less effective and increasing the risk of infectious disease. For meat-producing animals, oral administration has another advantage in that it avoids injection site reactions. Broken needles, contamination of the injection site, or the use of highly reactive adjuvants can induce abscesses that damage the carcass and the skins. These reactions decrease the value of the animal at slaughter. This is also an issue in fish vaccination programs where fish need to be harvested from their tanks or open sea cages and injected individually. Oral inoculation is quick and efficient and eliminates the need for multiple handling of animals to administer subsequent booster inoculations. Adverse immune reactions following oral administration are also much less likely to occur and are therefore safer.

Despite the advantages of administration, especially oral administration, of drugs, vaccines or pesticides, the development of the technology has been delayed by the lack of adequate delivery systems. In the absence of suitable delivery systems, most oral drugs, vaccines and pesticides undergo degradation in the gastrointestinal (GI) tract, especially under low-pH stomach conditions, resulting in limited absorption in the intestine with neutral pH, which in turn results in insufficient therapeutic effects, immune responses or pesticidal effects.

Various vehicles have been developed to deliver drugs, vaccines and pesticides to the gut-mucosal tissues. Biodegradable polymers, such as poly-(DL-lactide) and poly-(DL-lactide-co-glycolide), have been used to produce polymer particles for administration, especially oral administration, of drugs, vaccines or pesticides to animals. However, production of these polymer particles requires the use of solvents that can harm fragile drugs, vaccines or pesticides. Furthermore, the use of solvents prevents the incorporation of attenuated live organisms, such as viruses or bacteria, within those polymer particles.

Other challenges of developing adequate delivery systems include the need to select only suitable, for example, food or feed grade and biodegradable, compounds and adjuvants, and the need for a long-lasting and robust therapeutic, immunogenic or pesticidal effects.

### SUMMARY OF THE INVENTION

The invention provides a composition for delivering an active agent to an animal. The composition comprises an active agent, a first coating, a second coating, and a third coating. The active agent is coated with a first coating, which is coated with the second coating, which is coated with the third coating. The active agent is in contact with the first coating, not the second or third coating. The first coating separates the active agent from the second coating while the second coating separates the first and third coatings. The first, second and third coatings are different from each other, and each may be selected from the group consisting of an enteric polymer layer comprising one or more enteric polymers, a fat/protein layer comprising one or more fats, one or more proteins, or a combination thereof, and a mucoadhesive polymer layer comprising one or more mucoadhesive polymers. The active agent may be coated by the enteric polymer layer, the fat/protein layer, and the mucoadhesive polymer layer in any sequence or order. Depending the nature of the active agent and the other ingredients used in the composition, a specific sequence or order of the three layers may provide a more advantageous release profile or biological effect for the active agent in the animal.

A first optional composition according to the invention is Composition A, in which the first coating is the mucoadhesive polymer layer, the second coating is the enteric polymer layer and the third coating is the fat/protein layer.

A second optional composition is Composition B, in which the first coating is the mucoadhesive polymer layer, the second coating is fat/protein layer and the third coating is the enteric layer.

A third optional composition is Composition C, in which the first coating is the fat/protein layer, the second coating is the mucoadhesive polymer layer and the third coating is the enteric layer.

According to the present invention, the active agent may be in the form of a solution, dispersion or dry particles. The particles may have an average particle size in the range of about 0.1 μm-10 mm, preferably less than about 1 mm, more preferably less than about 500 μm.

The composition may be a liquid, solid or a slurry. The composition may be dry or wet.

The composition may comprise about 1-40% active agent. Aside from the active agent, the composition may comprise about 1-20% mucoadhesive polymer, about 1-30% enteric polymers, and about 1-40% fats, proteins or a combination thereof.

The active agent may be a bioactive agent. For example, the active agent is a therapeutic drug, an immunogen, an anti-viral, anti-bacterial, anti-fungal or anti-parasitical agent, or a pesticide. The pesticide may be a rodenticide.

Examples of the enteric polymers include alginate, ethyl cellulose, hydroxypropylmethylcellulose (HPMC), poly-(DL-lactide), poly-(DL-lactide-co-glycolide), and a mixture thereof.

The fat/protein layer may be a fat layer comprising one or more fats (without any protein), a protein layer comprising one or more proteins (without any fat), or a layer comprising one or more fats and one or more proteins. The fats may comprise hydrogenated or partially hydrogenated oils, coconut oils, palm oils, palm kernel oils, steric acid salts, waxes, or a mixture thereof. The proteins may comprise milk protein, gelatin, albumen, gluten, soy protein, zein or a mixture thereof.

The animal may be any insect or mammal. Examples of the insects include bed bugs, ants, fire ants, flies, mosquitos, fleas, spiders, ticks, beetles, cockroaches, termites, stink bugs mites and the like. The animal may be an aquatic animal, a terrestrial animal, or a mammal. The aquatic animal may be a fish, or a shellfish such as mollusks, crustaceans, and echinoderms. The mammal may be a primate such as human, dog, cat, horse, deer, bear, rodent, coyote, fox, squirrel, rabbit, raccoon, skunks and bats. The rodent may be a mouse or rat. The rodent may have a weight of at least about 50, 100, 150, 195, 250 or 500 grams. The animal may be a pest.

For each composition of the present invention, a preparation method is provided. The method comprises:

(a) mixing an effective amount of an active agent with a first coating to form a first coated product, wherein the active agent is coated with the first coating in the first coated product,

(b) mixing the first coated product with a second coating to form a second coated product, wherein the first coated product is coated with the second coating in the second coated product, and

(c) mixing the second coated product with a third coating to form a third coated product, wherein the second coated product is coated with the third coating in the third coated product, whereby the composition is prepared.

The preparation method of the present invention may further comprise drying the third layer coated product to form dry particles. The particles may have an average particle size in the range of about 0.1 μm-10 mm, preferably less than about 1 mm, more preferably less than about 500 μm. The particles may further comprise a surfactants, emulsifiers, preservatives and antioxidants.

Composition A may be prepared as follows:

(a) coating an effective amount of an active agent with a mucoadhesive polymer layer to form a first coated product, wherein the active agent is coated with the mucoadhesive polymer layer in the first coated product,

(b) coating the first coated product with an enteric polymer layer to form a second coated product, wherein the first coated product is coated with the enteric polymer layer, wherein the active agent is coated with the mucoadhesive polymer in the first layer and the enteric polymer in the second layer in the second coated product, and

(c) coating the second coated product with a fat/protein layer to form a third coated product, wherein the second layer coated product is coated with the fat/protein layer, wherein the active agent is coated with the mucoadhesive polymer in the first layer, the enteric polymer in the second layer, and the fat/protein in the third layer in the third coated product, whereby Composition A is prepared.

Composition B may be prepared as follows:

(a) coating an effective amount of an active agent with a mucoadhesive polymer layer to form a first coated product, wherein the active agent is coated with the mucoadhesive polymer layer in the first coated product,

(b) coating the first coated product with a fat/protein layer to form a second coated product, wherein the first coated product is coated with the fat/protein layer, wherein the active agent is coated with the mucoadhesive polymer in the first layer and the fat/protein in the second layer in the second coated product, and

(c) coating the second coated product with an enteric polymer layer to form a third coated product, wherein the second coated product is coated with the enteric polymer layer, wherein the active agent is coated with the mucoadhesive polymer in the first layer, the fat/protein in the second layer, and the enteric polymer in the third layer in the third coated product, whereby Composition B is prepared.

Composition C may be prepared as follows:

(a) coating an effective amount of an active agent with a fat/protein layer to form a first coated product, wherein the active agent is coated with the fat/protein layer in the first coated product,

(b) coating the first coated product with a mucoadhesive polymer layer to form a second coated product, wherein the second coated product is coated with the mucoadhesive polymer layer, wherein the active agent is coated with the fat/protein in the first layer and the mucoadhesive polymer in the second layer in the second coated product, and

(c) coating the second coated product with an enteric polymer layer to form a third layer coated product, wherein the second coated product is coated with the enteric polymer layer, wherein the active agent is coated with the fat/protein in the first a layer, the mucoadhesive polymer in the second layer, and the enteric polymer in the third layer in the third coated product, whereby Composition C is prepared.

A composition prepared according to the preparation method of the present invention is also provided.

The present invention also provides a method for providing controlled or targeted release of an active agent in an animal. The method comprises administering an effective amount of a composition comprising the active agent as described above. The method may further comprise releasing less than about 50%, 40%, 30%, 20%, 10%, 5% or 1% of the active agent in a gastric environment, for example, at a pH of about 0.1-3 or about 1-2, within the first 60, 30 or 15 minutes. The method may further comprise releasing at least about 50%, 60%, 70%, 80%, 90%, 95% or 99% of the active agent in an intestinal environment, for example, at a pH of about 5-8, about 5.5-7.5 or about 6.5-7.5, within the first 120, 60, 30 or 15 minutes. The method may further comprise keeping at least about 50%, 60%, 70%, 80%, 90%, 95%, 99%, for example, about 50-90%, of the active agent intact during passage in the animal stomach. The method may further comprise delivering at least 50-90% of the active agent to the intestine in the animal.

The active agent may be incorporated into a composition of the present invention having different combination of the first, second and third coating. Composition A, Composition B and Composition C may provide different release profiles for the same active agent. Composition A may provide better post-gastric delivery of the active agent than Composition B or C. For example, Composition A may provide less release of the active agent in a gastric environment and more release of the active agent in an intestinal environment as compared with Composition B or C. As a result, Composition A may be more effective than Composition B or C by, for example, at least about 10%, 20%, 30%, 40%, 50%, 60%, 70%, 80%, 90%, 95% or 99%, preferably at least about 20%, more preferably at least by about 50%, when the desirable site of action for the active agent is post-gastric in an animal. Composition C may provide a faster release profile in the intestinal and/or better absorption of the bioactive agent than Composition A or B. For example, Composition C may provide faster release of the active agent in an intestinal environment and more absorption of the active agent by the animal from the intestinal environment as compared with Composition A or B. As a result, Composition C may be more effective than Composition A or B by, for example, at least about 10%, 20%, 30%, 40%, 50%, 60%, 70%, 80%, 90%, 95% or 99%, preferably at least about 10%, more preferably at least by about 30%, when the bioavailability is low and the desirable site of action for the active agent is in the upper part of the animal intestine.

A method for post gastric delivery of an active agent to an animal is further provided. The delivery method comprises administering to the animal an effective amount of a composition of the present invention. At least about 50%, 60%, 70%, 80%, 90%, 95%, 99%, for example, about 50-90%, of the active agent may reach the intestine in an animal.

In some embodiments, more active agent reaches the intestine in an animal as formulated in Composition A than that in Composition B or C by, for example, at least about 10%, 20%, 30%, 40% or 50%, and/or more active agent is absorbed by the animal as formulated in Composition A than that in Composition B or C by, for example, at least about 10%, 20%, 30%, 40% or 50%.

In other embodiments, more active agent reaches the intestine in an animal as formulated in Composition B than that in Composition A or C by, for example, at least about 10%, 20%, 30%, 40% or 50%, and/or more active agent is absorbed by the animal as formulated in Composition B than that in Composition A or C by, for example, at least about 10%, 20%, 30%, 40% or 50%.

In yet other embodiments, more active agent reaches the intestine in an animal as formulated in Composition C than that in Composition A or B by, for example, at least about 10%, 20%, 30%, 40% or 50%, and/or more active agent is absorbed by the animal as formulated in Composition C than that in Composition A or B by, for example, at least about 10%, 20%, 30%, 40% or 50%.

A method for treating or preventing a disease or disorder (e.g., an infection) in an animal is provided. The treatment or prevention method comprises administering to the animal an effective amount of a composition of the present invention. The active agent is a therapeutic drug.

A method for vaccinating an animal (e.g., aquatic or terrestrial species) is also provided. The vaccination method comprises administering to the animal an effective amount of a composition of the present invention. The active agent is an antigen, which may be derived from an infectious microorganism, for example, a bacterium, fungus, virus or parasite. A specific protective immune response may be induced against the microorganism in the animal.

A method for controlling pests is further provided. The pest controlling method comprises administering to the pests an effective amount of a composition of the present invention. The active agent is a pesticide, for example, a rodenticide. The pests may be insects (e.g., ants, fire ants, cockroaches, flies, termites and the like) or rodents (e.g., mice and rats). The rodents may be large rats having an average weight of at least about 50, 100, 150, 195, 250 or 500 grams. The survival rate of the treated pests may be less than about 1%, 5%, 10%, 20%, 30%, 40%, 50% or 60%, preferably less than about 50%, more preferably less than 20%, most preferably less than 5%.

In some embodiments, the active agent as used in the methods of the present invention is more effective as formulated in Composition A than that in Composition B or C. For example, the therapeutic, immunogenic or pesticidal effect of the active agent as formulated in Composition A may be at least about 10%, 20%, 30%, 40% or 50% better than that in Composition B or C.

In other embodiments, the active agent as used in the methods of the present invention is more effective as formulated in Composition B than that in Composition A or C. For example, the therapeutic, immunogenic or pesticidal effect of the active agent as formulated in Composition B is at least about 10%, 20%, 30%, 40% or 50% better than that in Composition A or C.

In yet other embodiments, the active agent as used in the methods of the present invention is more effective as formulated in Composition C than that in Composition A or B. For example, the therapeutic, immunogenic or pesticidal effect of the active agent as formulated in Composition C is at least about 10%, 20%, 30%, 40% or 50% better than that in Composition A or B.

The term “an effective amount” refers to an amount of a composition comprising an active agent required to achieve a stated goal (e.g., controlled release of an active agent in an animal, treating or preventing a disease or disorder in an animal, vaccinating an animal, or controlling a pest). The effective amount of the composition comprising an active agent may vary depending upon the stated goal, the physical characteristics of the animal, the nature and severity of the disease or disorder, the existence of related or unrelated medical conditions, the nature of the active agent, the composition comprising the active agent, the means of administering the composition to the animal, and the administration route. A specific dose for a given animal may generally be set by the judgment of a physician or scientist. The composition may be administered to the animal in one or multiple doses. Each does may be at about 0.01-5000 mg/kg, preferably about 0.1-1000 mg/kg, more preferably about 1-500 mg/kg.

The composition of the present invention may be formulated for oral, sublingual, intranasal, intraocular, rectal, transdermal, mucosal, topical or parenteral administration. Parenteral administration may include intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids) injection or infusion. Any device suitable for parenteral injection or infusion of drug formulations may be used for such administration. For example, the composition may be contained in a sterile pre-filled syringe.

### DETAILED DESCRIPTION OF THE INVENTION

Unless otherwise defined herein, scientific and technical terminologies employed in the present disclosure shall have the meanings that are commonly understood and used by one of ordinary skill in the art. Also, as used herein and in the claims, the terms “at least one” and “one or more” have the same meaning and include one, two, three or more. Unless otherwise indicated, percentages or parts of components in compositions are on a weight basis. The term “dispersed” means suspended and/or dissolved.

“Crosslink” and variants thereof refers to the linking of two or more materials and/or substances, including any of those disclosed herein, through one or more covalent and/or non-covalent (e.g., ionic) associations. Crosslinking may be effected naturally (e.g., disulfide bonds of cystine residues) or through synthetic or semi-synthetic routes. Crosslinking of charged polymers can be effected by ionic association with a polyvalent counterion of opposite charge. Firm, solid structures, for example hydrogels, can be prepared by such crosslinking.

“Gastric protection” refers to the protection of a bioactive agent from gastric destruction and loss of activity.

“Being coated with” refers to a first material is surrounded by or embedded in a second material.

“An effective amount” refers to an amount of a composition comprising an active agent required to achieve a stated goal in an animal or animals (e.g., post-gastric delivery, treating or preventing a disease or disorder in an animal, or controlling pests). The effective amount of a composition comprising the active agent may vary depending upon the stated goals, the physical characteristics of the animal(s), the nature and severity of the disease or disorder (e.g., infection), existence of related or unrelated conditions, the nature of the active agent, the composition comprising the active agent, the means of administering the active agent to the animal(s), and the administration route. A specific dose for the animal(s) may generally be set by the judgment of a scientist, veterinarian or physician in the relevant field. The composition may be administered to the animal(s) in one or multiple doses.

The compositions of the invention include particulate materials comprising a bioactive agent having three layers of coating, comprising a mucoadhesive polymer layer, an enteric polymer layer and a fat/protein layer, which may be applied in any desirable sequence or order. The enteric polymer protects the bioactive from exposure to low pH conditions in the animal's stomach, since the polymer remains insoluble at low pH and remains intact as a protective coating or layer. The particles typically may have an average geometric size (sometimes referred to as diameter) in a range of 10 to 5000 micron, and may either be applied directly in that size range or reduced to that size by milling, grinding, or other means. Usually, the particles have a diameter of less than 1000 micron, preferably less than 500 micron.

In some embodiments, the mucoadhesive polymer and the bioactive agent are mixed together and in mutual contact, associated together within particles that are in turn coated with enteric polymer followed by a third coat of fats or proteins. The mucoadhesive polymer and/or the enteric coating polymer may be cross-linked or not.

In other embodiments, the bioactive agent is dispersed as above within oil droplets, and these are then coated with a mucoadhesive polymer. The resulting particles are in turn coated with enteric coating polymer. The mucoadhesive polymer and/or the enteric coating polymer may be cross-linked or not.

In some embodiments the invention provides a composition for oral administration to aquatic and terrestrial species of a bioactive agent against specific diseases. The composition comprises an effective amount of the bioactive agent. The present compositions are designed to present the bioactive material for contact with the gut mucosa of the animal to stimulate uptake and mucosal adhesion. Compositions according to this invention may be administered orally, typically with a feed or pharmaceutically acceptable carrier, including, for example, water (e.g., animal drinking water), tablets, capsules, bolus dosage forms, feed pellets or as a food additive to carry the composition into the gut of the targeted species.

The compositions of the invention provide several advantages in delivering a bioactive agent to an animal. First, the method of making the delivery system eliminates the use of organic solvents or high temperature and pH which are often required for the preparation of particles by other methods. By maintaining an aqueous environment at mild pH conditions and low temperatures throughout the preparation of the present composition, sensitive bioactives such as proteins, peptides, DNA and RNA fragments and antibiotic agents can be orally delivered. Second, the additional layer of enteric coating polymer protects the bioactive agent against degradation in the gastrointestinal tract. Third, the additional fats or proteins layer enclosing the bioactive agent, providing masking properties and preventing small bioactive molecules such as proteins, peptides and drugs from leaching to an aqueous environment during preparation, as well as during consumption and gastric exposure. Further, the delivery system can be easily formulated for efficient delivery to both aquatic and terrestrial species.

Preferably, all components used in preparing the inventive compositions are food grade, non-toxic and biodegradable, and naturally occurring. A description of materials useful for preparing the compositions follows.

Bioactive Agent

The bioactive agent may be a naturally occurring, synthetic, or semi-synthetic material (e.g., compounds, fermentates, extracts, cellular structures) capable of eliciting, directly or indirectly, one or more physical, chemical, and/or biological effects. The bioactive agent may be capable of preventing, alleviating, treating, and/or curing abnormal and/or pathological conditions of a living body, such as by destroying a parasitic organism, or by limiting the effect of a disease or abnormality. Depending on the effect and/or its application, the bioactive agent may be a pharmaceutical agent (such as a prophylactic agent or therapeutic agent), a diagnostic agent, and/or a cosmetic agent, and includes, without limitation, vaccines, drugs, prodrugs, affinity molecules, synthetic organic molecules, hormones, antibodies, polymers, enzymes, low molecular weight molecules proteinaceous compounds, peptides, vitamins, steroids, steroid analogs, lipids, nucleic acids, carbohydrates, precursors thereof, and derivatives thereof. The bioactive agent may also be a nutritional supplement. Non-limiting nutritional supplements include proteins, carbohydrates, water-soluble vitamins (e.g., vitamin C, B-complex vitamins, and the like), fat-soluble vitamins (e.g., vitamins A, D, E, K, and the like), minerals, and herbal extracts. The bioactive agent may be commercially available and/or prepared by known techniques.

Bioactive agents in the present invention include, without limitation, vaccines (vaccines can also be delivered as part of immune-stimulating complexes, conjugates of antigens with cholera toxin and its B subunit, lectins and adjuvants), antibiotics, affinity molecules, synthetic organic molecules, polymers, low molecular weight proteinaceous compounds, peptides, vitamins, steroids, steroid analogs, lipids, nucleic acids, carbohydrates, precursors thereof, and derivatives thereof. The bioactive agent may also be a pesticide, for example a rodenticide.

The bioactive agent may be an immunogen, i.e., a material capable of mounting a specific immune response in an animal. Examples of immunogens include antigens and vaccines. For example, immunogens may include immunogenic peptides, proteins or recombinant proteins, including mixtures comprising immunogenic peptides and/or proteins and bacteria (e.g., bacterins); intact inactive, attenuated, and infectious viral particles; intact killed, attenuated, and infectious prokaryotes; intact killed, attenuated, and infectious protozoans including any life cycle stage thereof, and intact killed, attenuated, and infectious multicellular pathogens, recombinant subunit vaccines, and recombinant vectors to deliver and express genes encoding immunogenic proteins (e.g., DNA vaccines).

The one or more bioactive agents may constitute at least 0.1% of the weight of the particles, excluding water, or at least 1%, or at least 5%. Preferably, they constitute at most 40%, or at most 20%, or at most 10%.

Mucoadhesive Polymer

The mucoadhesive polymer is a polymer that specifically binds to mucosal tissues, and helps retain the bioactive agent in close proximity to the mucosa, thereby improving administration. Suitable examples include synthetic polymers such as poly(acrylic acid), hydroxypropyl methylcellulose and poly(methyl acrylate), carboxylic-functionalized polymers, sulfate-functionalized polymers, amine-functionalized polymers, and derivatives or modifications thereof, as well as naturally occurring polymers such as carrageenan, hyaluronic acid, chitosan, cationic guar and alginate. Derivatized or otherwise modified versions of naturally occurring polymers may also be used, and many such polymers are known in the art. Nonlimiting examples include propylene glycol alginate and pectins, carboxymethyl chitosan, carboxymethyl chitosan, methyl glycol chitosan, trimethyl chitosan and the like.

A preferred mucoadhesive polymer is chitosan and modified or derivatized chitosan, which can be obtained through the deacetylation of chitin, the major compound of exoskeletons in crustaceans. Chitosan [a-(1˜4)-2-amino-2-deoxy-ß-D-glucan], a mucopolysaccharide closely related to cellulose, exhibits chemical properties that are determined by the molecular weight, degree of deacetylation, and viscosity. Chitosan can form microparticles and nanoparticles that can bind large amounts of antigens by chemical reaction with crosslinking agents such as phosphate ions, glutaraldehyde or sulfate ions.

Although chitosan is used in some preferred embodiments, other polymers may be used to achieve a similar mucoadhesive function. These include but are not limited to gelatin, alginate, dextran, hyaluronic acid, agar, and resistant starch.

The one or more mucoadhesive polymers may constitute at least 1% of the weight of the particles, excluding water, or at least 10%, or at least 15%. Preferably, they constitute at most 50%, or at most 30%, or at most 20%.

Fat Coating

In some traditional products, a significant amount of bioactive agent is lost to the aqueous environment by leaching out of the particle during its preparation and through the gastric passage, particularly small molecular size bioactive agents such as viruses, proteins, drugs, antibiotics, pesticides and the like. In the present invention, leaching of bioactive agent from the particle is largely eliminated by discrete particles, domains or phases containing the agent being dispersed in, or coated by, an oil. Any type of oil, including vegetable, animal or synthetic oils and fats in either liquid or solid form, or waxes, can be used for coating the bioactive agent. Vegetable origin oils used in the present invention include, without limitation, castor oil, coconut oil, coco butter, corn oil, cottonseed oil, olive oil, olive squalane, palm oil, peanut oil, rapeseed oil, safflower oil, sesame oil, soybean oil, sunflower oil, stearate, carnauba wax and mixtures thereof. Animal origin oils used in the present invention include, without limitation, fish oil, shark squalane, butterfat, beeswax, lanolin, lard and the like. In some cases the dispersing oil is a mixture of olive or shark squalanes with any other type of oil, fat or wax. The mass of oil may be greater than the combined mass of bioactive agent and mucoadhesive polymer.

Protein Coating

In some traditional products, a significant amount of bioactive agent is lost to the aqueous environment by leaching out of the particle during its preparation and through the gastric passage, particularly small molecular size bioactive agents such as viruses, proteins, drugs, antibiotics, pesticides and the like. In the present invention, leaching of bioactive agent from the particle is largely eliminated by discrete particles, domains or phases containing the agent being dispersed in, or coated by, protein. Any type of protein, including vegetable, animal or dairy proteins can be used for coating the bioactive agent. Vegetable origin proteins used in the present invention include, without limitation, soy protein, wheat protein, rice protein, pea protein and any other plant source of a protein. Animal origin proteins used in the present invention include, without limitation, fish proteins, meat and blood proteins Bovine and ova albumens and the like. Dairy origin proteins used in the present invention include, without limitation, any type of milk proteins including casein, whey protein, lacto-globulin and the like.

Enteric Coating Polymer

Mucoadhesive polymer coated particles with or without oil coating layer are coated with a layer of enteric coating polymer that provides gastric protection and post gastric release or delivery of the intact bioactive agent, i.e., release in the intestine.

Exemplary enteric coating polymers include polymers soluble in water at sufficiently high pH, but insoluble at low pH. They may be soluble at a pH greater than 5.0, and insoluble at a pH less than 4.0. Suitable polymers are substantially soluble or digestible under the relatively mild pH conditions of an animal's intestine, where the bioactive material is to be released, but insoluble and indigestible in the stomach, where the external matrix of enteric coating polymer protects the sensitive bioactive agent from deterioration. In some cases, the enteric coating polymer is cross-linked, for example with divalent cations, to prevent dissolution or digestion in the stomach.

Suitable enteric coating polymers can be selected from any of a wide variety of hydrophilic polymers including, for example, polyacrylic acid, poly(meth)acrylates, carboxymethyl cellulose, methyl cellulose, cellulose acetate phthalate and water soluble, natural or synthetic polysaccharide gums. One exemplary synthetic enteric coating polymer is EUDRAGIT® FS30D (Evonik Industries). Sodium alginate and pectins are preferred water soluble gums, because of their mild crosslinking conditions.

Alginates provide a preferred hydrophilic carrier matrix for gastric sensitive bioactive agents, particularly due to their ease of use in forming solid gel compositions. Alginate solutions form solid gels when combined or mixed with divalent cations. Nonetheless, in some embodiments the alginate is not cross-linked, but remains indigestible and insoluble in a gastric environment and therefore protective of the particle contents while under the low pH conditions of an animal's stomach.

Alginates comprise varying proportions of 1,4-linked β-D-mannuronic acid (M), a-L-guluronic acid (G), and alternating (MG) blocks. The viscosity of alginate solutions is mostly determined by the molecular ratio of M/G blocks. Low viscosity alginates may contain a minimum of 50% mannuronate units and their viscosity ranges from 20-200 mPa. Medium and high viscosity alginates contain a minimum of 50% of guluronic acid units and their viscosity may be over >200 mPas.

In some embodiments the enteric polymer is alginate, pectin or a mixture thereof. Low viscosity grade alginates and low methoxy pectins are preferred. Some low methoxy pectins have a methylation degree below 50%, and these may be cross-linked with a divalent cation such as Ba, Ca, Mg, Sr or Zn.

The one or more enteric coating polymers may constitute at least 10% of the weight of the particles, excluding water, or at least 20%, or at least 30%. They may constitute at most 70%, or at most 50%, or at most 40%.

Optional Ingredients

In some embodiments the composition optionally includes nutrients, nutraceuticals, feed attractants and/or taste masking compounds, in addition to the primary bioactive agent. Penetration enhancers or adjuvants may also be included.

Making the Compositions

In a typical procedure, a dry bioactive material in a powder form may coated with a layer of mucoadhesive polymer. Alternatively, an aqueous solution containing the bioactive agent is may be dissolved in a mucoadhesive polymer solution, optionally, the mucoadhesive polymer solution may be solidified by cross linking. For example, gelatin and agar polymers are solidified by dropping the temperature or changing the pH of the emulsion; while chitosan is solidified by raising the pH of the emulsion to above 6.5 and/or by adding counter ions such as sodium tripolyphosphate (TPP). The solidified cross linked solution is dried by freeze drying or spray drying to form a dry particulate material. The dry material is then coated with fat layer at a ratio of one part dry particle to 1.1-5 parts oil by weight until a uniform coat is produced. To assist in the formation of a uniform and stable coat, a nonionic surfactant may be added. Suitable nonionic surfactants, without limitation, include ethoxylated aliphatic alcohol, polyoxyethylene surfactants and carboxylic esters, etc.

A first general way of making the particles is as follows. A dry powder material comprising the bioactive agent is coated with mucoadhesive polymer solution and then dried to form bioactive agent particles having a first coating layer comprising a mucoadhesive polymer. The dry mucoadhesive coated bioactive is then coated with a second coat of fats typically, at a ratio of 1 part dry particles to 1.1-5 parts fats by weight. The fat coated particles then coated with a third layer of enteric polymer typically at a ratio of 0.5-1.5 parts of enteric polymer to 1 part of bioactive agent.

In a second general method, an aqueous mixture comprising a dispersed or dissolved bioactive agent and mucoadhesive polymer is spray dried or freeze dried to produce a dry mucoadhesive coated bioactive material. The dry material is then coated with a second layer of fats followed by a third layer of enteric polymer.

In a third general method, the mucoadhesive coated bioactive produced according to the first or the second general method described above is coated with a second coat of enteric polymer, followed by a third coat of fats.

In a fourth general method the fat layer according the above three general methods is mixed or replaced with a protein layer.

Using the Compositions

The compositions of this invention can be stored in aqueous suspension or dried by any drying method known in the art, and stored in a dehydrated state for long periods of time without a significant loss of activity.

Compositions according to the invention can be administered orally as a component of drinking water, as a food additive, or as part of a formulation containing a pharmaceutically acceptable carrier and optional adjuvants. Alternatively, the present compositions can be included in other standard oral dosage forms. Those skilled in the art will appreciate that there is a wide variety of art-recognized food, feed, nutraceutical or pharmaceutical dosage forms and acceptable carriers, suitable for delivering the composition to the targeted animal.

Administration of the compositions in accordance with this invention can be effected in single or multiple dose protocols. In one embodiment, bioactive compositions are administered in multiple dose protocols administered over a period of about 3 days to about 10 days or longer, and can be repeated periodically as the target species evidences loss of immunity.

For applications in drinking water for use in swine, poultry, cattle or aquatic animals, additional oil or inert polypropylene or polyester particles can be incorporated in the composition to increase buoyancy (i.e., decrease density) so that watering devices for delivery in fish culture tanks could be used to deliver the present compositions. Thus, the compositions can be administered to animals either as a component of their daily feed or as a component of their drinking water.

### EXAMPLES

### Example 1a

### Preparation of the Composition of the Invention

An inventive composition was prepared as follows. Three grams of mucoadhesive polymer (Chitosan, FMC Biopolymers Inc.) was dissolved in 100 ml of 0.5N glacial acetic acid solution at 50° C. The pH of the solution was adjusted to 5.8 with sodium hydroxide and the solution allowed to cool down to room temperature. Tween 80 (0.2%, Sigma, St Louis, Mo.) and Antifoam (0.5%, Sigma, St Louis, Mo.) were added and the chitosan solution kept at 4° C. until use. A 30 ml solution containing 300 mg ovalbumin (“OVA”, a model vaccine) was added to the chitosan solution to produce a mixture. The resulting solution was added to 195 g olive oil containing 5% Span-80 (Sigma) and homogenized at 10,000 rpm for 30 min in an ice bath to form a water in oil emulsion. A 20 ml aqueous sodium tripolyphosphate (5%) and 0.5N NaOH was slowly added with mixing to the bioactive agent emulsion containing ovalbumin and cross-linked chitosan microparticles in a continuous oil phase. The particles were allowed to harden for at least 2 h but not removed from the oil phase.

The dispersion of particles in oil was stirred into 330 ml of a 9% aqueous solution of low viscosity grade sodium alginate (FMC Biopolymers Inc.) that also contained 66 g oligosaccharides (instant inulin, Cargill, Minneapolis, Minn.), 10 g lecithin and 3 g Tween-80. The resulting aqueous dispersion was injected into a cross-linking solution containing 5% CaCl₂ to form alginate matrix beads, each containing multiple oil droplets that in turn each contained microparticles of ovalbumin and cross-linked chitosan. The beads were freeze dried and milled below 150 μm sized particles to obtain a dry composition of the present invention.

### Example 1b

An alternative method of forming compositions of the invention utilizes an emulsion of an aqueous bioactive solution in an oil. Ten ml of an aqueous solution containing 100 mg ovalbumin was combined with 15 g canola oil containing 5% Span-80 and homogenized to form a fine water in oil emulsion. The emulsion was mixed with a 100 ml of 3% aqueous chitosan solution, and the dispersion was injected into a cross-linking solution containing 5% tripolyphosphate solution (5% TPP). The particles were allowed to harden for at least 2 h. The resulting solid cross-linked chitosan particles contained embedded oil droplets, and each of these oil droplets in turn contained dispersed smaller than 10 μm droplets of the aqueous ovalbumin. The solid particles were isolated by filtration and were finely dispersed in 400 ml of an aqueous solution of 9% low viscosity grade alginate. The resulting aqueous dispersion was injected into a cross-linking solution containing 5% CaCl₂ to form alginate matrix beads. The beads were freeze dried and milled below 150 μm sized particles to obtain a dry composition of the present invention.

### Example 2

### Preparation of an Immunogenic Composition

Chitosan (3 g, FMC Biopolymer) was dissolved in 100 ml solution of 0.5N glacial acetic acid at 50° C. The pH of the solution was adjusted to 5.8 with sodium hydroxide and the solution was allowed to cool to room temperature. A 10 ml solution containing 100 mg ovalbumin (OVA) as a model vaccine was mixed with 50 mg of immune-stimulating agent (beta glucan, AHD International, Atlanta, Ga.) and added into the chitosan solution. The resulting mixture was emulsified in 150 g shark squalane oil (Jedwards International) containing 5% w/w Span-80 at 10,000 rpm for 30 minutes to form an emulsion of aqueous droplets of OVA, chitosan and beta glucan in a continuous oil phase. The emulsion was added with stirring to 400 ml of an aqueous solution of 9% low viscosity grade sodium alginate in 0.5N NaOH that also contained oligosaccharides (40 g, instant inulin). The resulting emulsion was injected into a 5% CaCl₂ solution to crosslink the alginate, resulting in an immunogenic composition of the current invention. The composition was freeze dried and milled to particles less than 250 μm in size.

### Example 3

### Preparation of a Composition for Treatment/Prevention of Parasitic Infection of Fish

A composition containing a protein antigen or parasiticidal compound for treatment of parasite infestation in fish is prepared. Ten mg of the bioactive agent is dissolved in 10 ml of 3% aqueous chitosan solution as described in Example 2 above, and emulsified in 15 g of oil mixture containing 75% olive oil, 20% squalane oil and 5% Span-80.

One ml of an aqueous 5% sodium tripolyphosphate, 0.5N NaOH solution is emulsified in one g olive oil and mixed into the bioactive agent emulsion, resulting in a dispersion in oil of particles containing the bioactive agent and cross-linked chitosan. The dispersion is allowed to stand for 2 h to harden the cross-linked chitosan. The resulting dispersion of particles in oil is added with stirring to a 20 ml solution containing 9% low viscosity grade sodium alginate, 1% low methoxypectin, 30% w/w instant inulin and 1% Tween-80. The resulting mixture is injected into a cross-linking solution containing 3% CaCl₂ to form beads of an alginate-pectin matrix containing embedded dispersed oil droplets each in turn containing microparticles of bioactive and cross-linked chitosan. The beads are freeze dried and milled to below 150 μm to obtain a dry composition of the present invention.

### Example 4

### Preparation of a Composition Containing a Pharmaceutical Drug

A composition containing a pharmaceutical drug (a glucocorticoid such as dexamethasone or methyl prednisolone) for treatment of colonic diseases is prepared. The drug is added to chitosan solution as described in Example 1 or 2 above, and emulsified in a mixture of 95% squalane oil and 5% Span-80. An alkali emulsion containing 5% sodium tripolyphosphate in 0.5N NaOH in squalane oil is prepared and slowly mixed (20% w/w) into the bioactive emulsion to crosslink the chitosan, and the mixture is allowed to stand for at least 2 h to harden the cross-linked particles. The oil dispersion of chitosan microparticles is mixed into a liquid containing the enteric coating polymer (30% w/w EUDRAGIT® FS30D, Evonik Industries) at a ratio of 1:3 emulsion/Eudragit liquid and spray-dried to form a dry particulate composition of the present invention.

### Example 5

### Encapsulation Efficiency of a Bioactive Agent in the Composition of the Current Invention

The effect of the additional oil dispersion and enteric coating polymer matrix in the composition of the current invention was evaluated using ovalbumin (OVA) to simulate a typical protein drug or vaccine. Three OVA (Sigma) containing compositions were prepared. Composition 1 consisted of OVA bound chitosan microparticles, prepared by dissolving 100 mg OVA in 10 ml of 3% chitosan solution and injecting the solution into 10% aqueous TPP to form cross-linked beads, followed by a 2 h hold to harden the beads and subsequent freeze drying and milling. Composition 2 was made by emulsifying a 10 ml aqueous solution containing 100 mg OVA in 15 g of squalane oil containing 3% Span-80, and mixing the resulting emulsion in 20 ml of 3% chitosan solution. The resulting slurry was then injected into a 10% TPP solution to form beads, followed by hardening, freeze drying and milling as above. Composition 3 consisted of OVA bound chitosan microparticles according to the invention, prepared as in Example 2.

The encapsulation efficiency of OVA in the three types of composition was determined as follows. Five hundred mg of each composition was dispersed in 10 ml RIPA buffer and incubated at room temperature for 30 min. The suspensions were vortexed for 5 min and then centrifuged at 3000 rpm for 15 min. The supernatant was assayed for OVA content using Western Blot analysis, as follows.

Western Blot: the compositions were lysed with RIPA buffer as described above, and a calculated amount equivalent to 12 μg of protein per sample was loaded on a 10% SDS-polyacrylamide gradient gel (SDS-PAGE, Bio-Rad, Hercules, Calif.). Proteins were transferred onto a PVDF membrane (Bio-Rad) and blocked for 1 h with 5% non-fat milk in PBS containing 0.5% Tween-20 (PBS-T). Blots were incubated with an appropriate primary antibody at 1:5000 dilutions for 1 h at room temperature. After washing with PBS-T (3×10 mL, 5 min. each), the membranes were incubated with an appropriate HRP-conjugated secondary antibody (EMD Millipore Corporation, Billerica, Mass., USA) at 1:5000 dilution for 1 h. After washing with PBS-T (3×10 mL, 5 min. each), chemiluminescent films were developed with an ECL substrate (Amsheram Biosciences). The encapsulation efficiency of OVA (% retention of the original amount of OVA) is presented in Table 1.

TABLE 1

| Composition | Encapsulation Efficiency (%) |
|---|---|
| 1 | 70 |
| 2 | 95 |
| 3 | 95 |

The results demonstrate the protective effect of the oil dispersion in compositions 2 and 3 in preventing the leaching (loss) of the bioactive agent to a simple aqueous environment. However, significant differences between comparative Composition 2 and inventive Composition 3 were found when tested under gastric conditions, as described below in Example 7.

### Example 6

### Degradation of Unprotected Protein Antigen Activity in Simulated Gastric Juice

To evaluate the loss of activity of a protein antigen following a typical gastric exposure, non-encapsulated OVA (10 mg) was incubated in 10 ml simulated gastric fluid containing 0.08% pepsin at pH-2 for 2 h at 37° C. on a shaker. Medium was withdrawn at 15 min, 30 min, 60 min and 120 min incubation times, and the amount of residual OVA was analyzed using Western Blot analysis as described above. Table 2 shows the degradation of OVA over 2 h exposures in simulated gastric juice, indicated as % remaining activity relative to pre-exposure activity.

TABLE 2

| Time (min) | Remaining activity (%) |
|---|---|
| 15 | 61 |
| 30 | 55 |
| 60 | 38 |
| 120 | 2 |

These results demonstrate that the activity of unprotected protein-based antigen or bioactive agent will be completely degraded in the animal digestive tract.

### Example 7

### Gastric Protection of a Bioactive Agent in the Composition of the Current Invention

To evaluate the remaining activity of a protein antigen after gastric exposure, three compositions were prepared as described in Example 5. Five hundred mg each of the three compositions were incubated in 10 ml simulated gastric fluid containing 0.08% pepsin at pH-2 for 2 h at 37° C. on a shaker. At the end of 2 h exposure, the gastric solutions were withdrawn and the remaining activity of the OVA in the compositions was measured as described in Example 5. Table 3 shows the remaining activity of OVA in each of the compositions after 2 h exposure to simulated gastric juice.

TABLE 3

| Composition | Remaining activity (%) |
|---|---|
| 1 | 10 |
| 2 | 20 |
| 3 | 90 |

These results clearly demonstrate the superior gastric protective effect of inventive Composition 3 relative to prior art Compositions 1 and 2.

### Example 8

### The Effect of the Viscosity Grade of Alginate in the Composition on Gastric Protection

Three compositions containing 9% low grade viscosity alginate (50 cP), 6% medium grade viscosity alginate (300 cP) and 1% high grade viscosity alginate (800 cP) were prepared according to Example 2 above. The three compositions were exposed to simulated gastric juice as described in Example 7 and the remaining activity of the OVA in the compositions measured as described in Example 5. Table 4 shows the remaining activity of OVA in each of the compositions after 2 h exposure to simulated gastric juice.

TABLE 4

| Alginate viscosity grade | Remaining activity (%) |
|---|---|
| High (800 cp) | 25 |
| Medium (300 cp) | 40 |
| Low (50 cp) | 90 |

These results demonstrate that compositions containing lower viscosity grade alginate provide higher protection of a protein-based antigen or bioactive in the simulated animal digestive tract.

### Example 9

### Optimal Particle Size of the Inventive Composition

In this example the protecting effect of the particle size of a dried and milled inventive composition in a simulated gastric environment was assessed. An OVA composition was prepared as described in Example 5, followed by separating the dry powder into 2 particle sizes: small particles that went through a 50 μm screen, and large particles that were captured on the 50 μm screen but passed through a 100 μm screen. Table 5 shows the remaining activity of OVA in each particle size of the composition after 2 h exposure in simulated gastric juice.

TABLE 5

| Particle size | Remaining activity (%) |
|---|---|
| 50-100 μm | 90 |
| <50 μm | 40 |

These results show that optimal gastric protection is provided when the dry composition is milled to a particle size above 50 μm.

### Example 10

### Oral Administration of OVA Composition to Mice

Ovalbumin is orally administered to mice to test the efficacy of the inventive compositions in inducing an immune response.

Animals: Ten-twelve week old female BALB/C mice are used. Mice are fed ad libitum. Each experimental group is housed in a separate cage.

Ovalbumin composition: Ovalbumin (1 mg/g of ovalbumin, Sigma, St. Louis, Mo.,) is incorporated into the inventive composition as described in Example 5. Three groups of 4 mice each are inoculated as follows: 1) ovalbumin (OVA) in the composition, administered orally, 2) OVA solution, administered subcutaneously (SC), 3) antigen free composition administered orally. Mice are inoculated at 0 and 3 weeks. Each dose administers a total 100 mg of dry composition mixed with corn oil at a ratio of 1:2 w/w of dry composition/oil, coated onto feed pellets. At week 4 each mouse is euthanized and serum and spleen cells are harvested.

Immunological assays: Serum is assayed for IgG and IgA by ELISA. ELISA is performed using OVA absorbed to polystyrene plates. Samples are placed in wells in triplicate at a 1:25 dilution for serum. Goat anti-mouse antibody conjugated with horse radish peroxidase is used, followed by an orthophenylenediamine substrate (Sigma, St. Louis, Mo., U.S.A.). Optical density of each well is determined by placing the plate in a microtiter plate spectrophotometer and reading the plate at 490 nm. Spleen cells are tested for antibody secreting cells (ASC) specific for OVA, using techniques described previously.

The OVA specific IgG and IgA antibodies are quantified by determining the increase in optical density over time. OVA specific serum and IgA IgG and ASC secreting cells for each mouse inoculated with OVA are expected to be equally increased in those mice injected with OVA and orally fed the composition of the present invention. No OVA specific IgG or IgA antibodies are expected to be detected in mice fed antigen free composition. Thus, the composition is expected to be effective in inducing an immune response upon oral administration.

### Example 11

### Oral Administration of a Composition Containing Antigens to Chickens

Salmonella enteritidis is a major cause of disease in laying hens. Infection decreases production and increases mortality in flocks. Moreover, S. enteritidis can be passed through the egg to baby chicks, infecting subsequent generations or humans who consume infected eggs. Since infection begins by this bacteria attaching and invading the intestinal mucosa, and long term infection involves infection of intestinal lymphoid tissues, stimulation of mucosal immunity is imperative to control this disease.

To assess the efficacy of vaccinating chickens with the vaccine compositions of the present invention, the flagellin of Salmonella enteritidis, a key immunogen, is incorporated within the composition according to Example 2, except that the vaccine emulsion is mixed in the alkaline sodium alginate phase at a ratio of 1:2 w/w and the slurry is spray-dried. The dry composition is top-coated on feed and administered orally to chicks. Ten-week old chickens receive 3 oral doses at 2 week intervals of the composition loaded with either 300 μg of flagellin antigen of S. enteritidis or Bovine serum albumin. One week after the last oral dose of antigen, serum and intestinal fluid are collected and assayed for flagellin specific antibodies by ELISA. Results are expected to show that orally vaccinated birds have significantly increased flagellin specific antibodies in the serum.

### Example 12

### Oral Administration of a Composition Containing Antigens to Calves

The efficacy of orally administered ovalbumin containing composition prepared in accordance with the present invention to stimulate an immune response in the lungs of calves is demonstrated.

Ovalbumin is incorporated in the composition as described in Example 1a. For oral administration to calves, a composition containing a dose of 40 μg of ovalbumin per mg is administrated in the feed. Four calves are used per experimental group and each calf receives 5 mg of ovalbumin per dose for 5 consecutive days.

Two groups of calves are used to assess the efficacy of orally administered ovalbumin to induce a specific immune response. Group 1 is given 2 doses of ovalbumin in an incomplete Freund's adjuvant by subcutaneous (SC) injection 3 weeks apart. This group serves as the parenteral control, the method of vaccination routinely used for any vaccine. Group 2 receives 2 oral regimens of a composition containing ovalbumin 3 weeks apart. Serums are evaluated for isotypic antibody response to ovalbumin. Results are expected to show that a significant amount of OVA specific IgG and IgA is produced in the orally fed calves with OVA-containing composition. The expected very high level of serum IgA predicts high effectiveness in stimulating a systemic immune response in cattle.

### Example 13

### Oral Administration of a Composition Containing Vibrio Antigens to Fish

Vibrio alginolyticus is a serious bacterial infection in aquaculture, particularly severe in rainbow trout. It is now endemic in all trout-producing countries where it can cause severe economic losses. It is also becoming a more significant pathogen of farmed salmon, primarily in the freshwater growing phase, but it has been reported to cause losses in the sea as well. Vaccination can prevent V. alginolyticus from having a significant impact at any stage of the farming cycle of salmonids. A typical vaccination program involves a primary vaccination of fry of 2-5 grams and an oral booster vaccination 4-6 months after the primary vaccination. However, an ideal vaccination program would involve only one type of vaccination provided periodically to the fish in order to maintain an effective antibody titer in the fish serum throughout the entire culture period.

Experimental design: The efficacy of orally administered ERM vaccine containing composition prepared in accordance with the present invention to stimulate an immune response in trout serum is demonstrated.

Attenuated V. alginolyticus is incorporated within a composition as described in Example 1b. For oral administration to fish, a composition containing a dose of 2 μg of V. alginolyticus vaccine per mg is administrated in the feed. Twenty fish at an average size of 5 g are used per experimental group and each fish receives 1 dose of V. alginolyticus vaccine in feed ration for 5 consecutive days.

Three groups of fish are used to assess the efficacy of orally administered V. alginolyticus vaccine to induce an immune response relative to a standard vaccination by injection. Group 1 is vaccinated using a vaccination by injection protocol. This group serves as the parenteral control, the method of vaccination routinely used for any vaccine. Group 2 receives one oral regimen of a composition containing V. alginolyticus vaccine. Group 3 receives one oral regimen of vaccine free composition. Sera are evaluated for isotypic antibody response to V. alginolyticus 6 weeks post vaccination. Results are expected to show that a significant amount of V. alginolyticus specific IgA is produced in the orally fed fish with V. alginolyticus-containing composition. The immune response in serum of both orally and injected vaccinated fish is expected to be comparable. The expected very high level of serum IgA predicts high effectiveness in stimulating a systemic immune response in fish.

### Example 14

### Compositions Containing Rodenticide

a) This example describe the preparation of a dry composition containing a parasiticidal compound coated with fats that serves as masking agent and to improve palatability of the animal feed followed by an additional coat of enteric polymers. A 350 g mixture (1:1 w/w ratio) of CEBES 27-70 (Aarhus United USA Inc., Port Newark, N.J. and 17-Stearine (Lodres Croklaan N. America LLC., Channahon, Ill.) was melted at 45° C. 350 g of rodenticide in a form of dry powder was slowly mixed in the molten fat at 45° C. and under agitation. The material was allowed to cool to room temperature while agitation continued and the final particulate material was frozen then milled and sieved through 400 micron sieve. The harden bioactive fat mixture was first coated with 350 g of 9% low viscosity alginate (Sigma) solution under agitating to form a deposition of alginate layer on the particulate powder. The material was air dried and sieved through 500 micron sieve. 1050 g of Eudragit FS30D (Evonik Industries, Essen Germany) was slowly sprayed on the agitated material and the material air dried and sieved through 600 micron sieve.

b) A second example describes a preparation of a dry composition containing an antigen vaccine or parasiticidal compound coated first with enteric polymers followed by an additional coat of fats that serves as masking agent and to improve palatability of the animal feed. 350 g of rodenticide in a form of dry powder was mixed with 350 g of 9% low viscosity alginate (Sigma) solution under agitating to agglomerate and form a deposition of alginate layer on the particulate powder. The material was air dried and sieved through 250 micron sieve. 1050 g of Eudragit FS30D (Evonik Industries, Essen Germany) was sprayed on the agitated material and the material air dried and sieved through 400 micron sieve. A 320 g mixture (1:1 w/w ratio) of CEBES 27-70 (Aarhus United USA Inc., Port Newark, N.J. and 17-Stearine (Lodres Croklaan N. America LLC., Channahon, Ill.) was melted at 45° C. and then slowly mixed in the dry powder at 45° C. and under agitation. The material was allowed to cool to room temperature while agitation continued and the final particulate material sieved through 600 micron sieve.

c) A third example describes a preparation of a dry powder of an antigen vaccine or a parasiticidal compound coated with enteric polymers followed by an additional coat of proteins that serves as masking agent and to improve palatability of the animal feed. 350 g dry rodenticide in a form of dry powder was mixed with 350 g of 9% low viscosity alginate (Sigma) solution under agitating to agglomerate and form a deposition of alginate layer on the particulate powder. The material was air dried and sieved through 250 micron sieve. 1050 g of Eudragit FS30D (Evonik Industries, Essen Germany) was sprayed on the agitated material and the material was air dried and sieved through 400 micron sieve. A 2000 g solution containing 16% Caseinate (Sigma) was then applied in the same manner as described with the Eudragit while agitation continued and the final particulate material air dried and sieved through 600 micron sieve.

### Example 15

### Mice Infestation Control

Warfarin is the most common rodenticide used to control rat and mouse infestations. Rodents ingesting baits containing Warfarin exhibit obvious symptoms of poisoning in 15-30 minutes and become unconscious in 1-2 hours. However, because of its fast acting effect the rodent typically ingest a sub lethal amount of Warfarin and recovery occurs within 8 hours. Encapsulating the Warfarin may delay onset of the symptoms, allowing for the consumption of a full lethal dose.

Experimental Methods: Ten-twelve week old female BALB/C mice are used. Mice are fed ad libitum. Each experimental group is housed in a separate cage.

Inventive Warfarin composition: Warfarin (400 mg/g of composition, Sigma, St. Louis, Mo.) is incorporated into a composition as described generally in Example 14a. Three groups of 4 mice are each fed ad libitum as follows: 1) Inventive Warfarin composition, mixed in baits at 4% Warfarin activity, 2) Unencapsulated Warfarin, mixed in baits feed at 4% activity, 3) Bait containing a composition as in Example 14a, containing no Warfarin or other bioactive. The feed intake and kill effect on the mice are monitored.

Results show that feed intake of groups 1 and 3 are similar while the feed intake in group 2 (unencapsulated Warfarin) is over 25% less. It is expected that all mice in group 1 are dead after 8 h from feeding while all group 2 mice remain alive after 8 h from feeding.

### Example 16

### Rat Infestation Control

Experimental Methods: 250-500 g rats are used. Rats are fed ad libitum. Each experimental group is housed in a separate cage.

Inventive Warfarin composition: Warfarin (400 mg/g of composition, Sigma, St. Louis, Mo.) is incorporated into a composition as described generally in Example 14b. Three groups of 4 rats are each fed ad libitum as follows: 1) Inventive Warfarin composition, mixed in baits at 4% Warfarin activity, 2) Unencapsulated Warfarin, mixed in baits feed at 4% activity, 3) Bait containing a composition as in Example 14b, containing no Warfarin or other bioactive. The feed intake and kill effect on the rats are monitored.

Results show that feed intake of groups 1 and 3 are similar while the feed intake in group 2 (unencapsulated Warfarin) is over 25% less. It is expected that all rats in group 1 are dead after 8 h from feeding while all group 2 rats remain alive after 8 h from feeding.

### Example 17

### Gastric Protection of Model Protein

This example describes the release profile in simulated gastric juice of a protein (BSA, Sigma). The protein was encapsulated as described in example 15a using two enteric coating polymers having different pH release profile (Eudragit™ FS30D and L30D55). In vitro release studies were performed using i.e., simulated gastric fluid (pH=2). 0.5 g of the encapsulated protein samples were suspended in 10 ml of simulated gastric fluid and incubated for 2 h at 37° C. on a rotary shaker at 150 rpm. Samples were withdrawn after 30 min and 120 min incubation for spectrophotometric reading at 595 nm. Results in Table 6. demonstrate that a bioactive having a second coating of high pH release Eudragit FS30D (˜pH6.5) was protected significantly better in gastric exposure than a bioactive having a second coating of low pH release Eudragit L30D55 (˜pH 5.5).

TABLE 6
Release profile of a model protein in simulated gastric juice

| | Gastric Release | Gastric Release |
|---|---|---|
| Formulation | (T = 30 min) | (T = 120 min) |
| Eudragit FS30D | 1.5% | 16.04% |
| Eudragit L30D55 | 19.4% | 49.00% |

### Example 18

### Gastric and Intestinal Release of the Model Protein

This example describes the release profile in simulated gastric and intestinal juices of a protein (BSA, Sigma). The protein was encapsulated as described in Example 15a using two enteric coating polymers having different pH release profile (Eudragit™ FS30D and L30D55) as described in Example 16. In vitro release studies were performed using simulated gastric and intestinal fluid (pH=2 and pH=6.8, respectively). 0.5 g of the encapsulated protein samples were suspended in 10 ml of simulated gastric juice for 30 or 120 min followed by a respectively same incubation period in intestinal fluid. All incubations were done at 37° C. on a rotary shaker at 150 rpm. Samples were withdrawn after 30 min and 120 min incubation for spectrophotometric reading at 595 nm. Results in Table 7. demonstrate that a bioactive having a second coating of high pH release Eudragit FS30D (˜pH6.5) was gradually released over the 2 h exposure in the intestine while most of the bioactive having a second coating of low pH release Eudragit L30D55 (˜pH 5.5) was released within the first 30 min of exposure in the gastric. Overall, results in Examples 2 and 3 show that the composition of the present invention is effectively protecting a bioactive in the animal gastric environment with less than 20% loss of activity, whereas in the intestinal environment it was fully released.

TABLE 7
Release profile of a model protein in simulated gastric juice and intestinal
juice

| Formulation | (T = 30 min) | (T = 120 min) |
|---|---|---|
| Eudragit FS30D | Gastric - 1.5% | Gastric - 19.04% |
| | Intestinal - 41.5% | Intestinal - 75.09% |
| Eudragit L30D55 | Gastric - 16.04% | Gastric - 49% |
| | Intestinal - 58.3% | Intestinal - 37.5% |

### Example 19

### Optimization of the Layer Thickness of the Second Coat

To evaluate the effect of layer thickness of the second coating on gastric and intestinal release, Composition were prepared as described in example 15a with either 50% or 100% coating of Eudragit FS30D. In vitro release studies were conducted as described in Examples 16 and 17. Results in Table 8. show the release profile of the bioactive from a composition having 100% or only 50% of a second coating. It shows that a favorable release profile may be better achieved at lower amount of enteric polymer coating.

TABLE 8
Release profile of a model protein in simulated gastric and intestinal juice,
as a function of the amount of the enteric coat material in the composition.

| | Gastric Release | Intestinal release |
|---|---|---|
| Eudragit FS30D | (T = 30 min) | (T = 30 min) |
| 100% coating | 1.5% | 41.5% |
| 50% coating | 22.1% | 80% |

### Example 20

### Stability of the Composition During Feed Pelleting Exposure

Encapsulated model protein was prepared as described in example 15a. The composition was subjected to simulated industrial feed pelleting conditions by incubation for 5 min at 80° C. in a water bath, followed by tableting in a tablet press under a pressure of 1 ton. The tableted composition was crushed suspended in 10 ml of water and the amount of the released protein evaluated by a spectrophotometer at 595 nm. It was found that 68.4% of the protein remained intact in the composition following the harsh pelleting exposure.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

All documents, books, manuals, papers, patents, published patent applications, guides, abstracts, and other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed:

1. A method for preparing a composition, comprising:
(a) mixing an effective amount of an active agent with a first coating to form a first coated product, wherein the active agent is coated with the first coating in the first coated product,
(b) mixing the first coated product with a second coating to form a second coated product, wherein the first coated product is coated with the second coating in the second coated product, and
(c) mixing the second coated product with a third coating to form a third coated product, wherein the second coated product is coated with the third coating in the third coated product, whereby the composition is prepared, wherein the active agent is in contact with the first coating, not the second or third coating, wherein the first coating separates the active agent from the second coating, wherein the second coating separates the first and third coatings, wherein the first, second and third coatings are different from each other, and wherein each of the first, second and third coatings is selected from the group consisting of (a) an enteric polymer layer comprising one or more enteric polymers, (b) a fat/protein layer comprising one or more fats, one or more proteins, or a combination thereof, and (c) a mucoadhesive polymer layer comprising one or more mucoadhesive polymers.

2. The method of claim 1, further comprising drying the third layer coated product to form dry particles, wherein the dry particles have an average particle size in the range of about 0.1 μm-10 mm.

3. A composition prepared by the method of claim 1.

4. A method for providing controlled release of an active agent in an animal, comprising administering to the animal an effective amount of the composition of claim 3.

5. The method of claim 4, further comprising releasing less than 50% of the active agent in a gastric environment having a pH of about 0.1-3 within 60 minutes.

6. The method of claim 4, further comprising releasing at least 50% of the active agent in an intestinal environment having a pH of 5-8 within 120 minutes.

7. The method of claim 4, further comprising keeping at least 50-90% of the active agent intact during passage in the animal stomach.

8. The method of claim 4, further comprising delivering at least 50-90% of the active agent to the intestine in the animal.

9. A method for treating or preventing a disease or disorder in an animal, comprising administering to the animal an effective amount of the composition of claim 3, wherein the active agent is a therapeutic drug.

10. A method for vaccinating an animal, comprising administering to the animal an effective amount of the composition of claim 3, wherein the active agent is an antigen.

11. The method of claim 10, wherein the antigen is derived from an infectious microorganism selected from the group consisting of a bacterium, a fungus, a virus and a parasite, whereby a specific protective immune response against the microorganism is induced in the animal.

12. A method for controlling a pest, comprising administering to the pest an effective amount of the composition of claim 3, wherein the active agent is a pesticide.

13. The method of claim 12, wherein the pest has a survival rate of less than 5%.
